(19) United States
(12) Patent Application Publication
(10) Pub. No.: US 2023/0181924 A1
(43) Pub. Date: Jun. 15, 2023

(54) APPARATUS AND SYSTEM FOR TREATING SKIN LESIONS AND EVALUATING THE EFFECTIVENESS OF SAID TREATMENT

(71) Applicant: Zerigo Health, Inc., San Diego, CA (US)

(72) Inventor: Frank D'Amelio, San Clemente, CA (US)

(21) Appl. No.: 17/547,216

(22) Filed: Dec. 9, 2021

(51) Int. Cl. A61N 5/06; G06T 3/40; H04N 7/18

(52) U.S. Cl. CPC A61N 5/0616; G06T 3/4038; H04N 7/18; A61N 2005/0652; A61N 2005/0661

(57) ABSTRACT

An apparatus for treating a lesion within a skin region includes a light emitting diode (LED) array configured to treat the lesion and a camera capable of imaging the skin region. The LED array includes one or more LEDs, and the camera includes an image sensor and a lens. The apparatus is configured to automatically photograph the skin region before and/or after the lesion treatment. A system for treating a lesion within a skin region includes the apparatus and a remote device.

### CROSS-REFERENCE TO OTHER APPLICATIONS

This application refers to U.S. application Ser. No. 17/325,203, filed May 19, 2021, which is assigned to the current Applicant. This application is being filed on the same day as the application identified by Attorney Docket Number 27471.0003, also assigned to the current Applicant. Each of these applications is herein incorporated by reference in its entirety.

### BACKGROUND

One set of ailments afflicting people are skin lesions. One method for treating skin lesions involves applying light transmitted from light-emitting diodes (LEDs) to the skin lesions. Patients who are being treated for skin lesions as well as their healthcare providers (and maybe even insurance companies) often want to know if the treatment is working. A treatment often does not effect immediate relief, so it is difficult to evaluate over the course of time whether the lesions are decreasing in size and/or severity. Patients and/or healthcare providers often take “before” and “after” (sometimes referred to as “comparison”) images of the lesions to determine the size or surface area before and during (the “after” images) treatment. Typically, these comparison images suffer from differing distances and/or magnifications, differing lighting and/or color temperatures, poor focus, and differing image angles.

### BRIEF DESCRIPTION OF THE DRAWINGS

FIG. 1A is a diagram showing an LED-based skin treatment apparatus with a camera, according to an embodiment of the present invention;

FIG. 1B is a diagram showing an LED-based skin treatment apparatus with a camera and a display screen, according to an embodiment of the present invention;

FIGS. 2A-2B are diagrams showing the LED-based skin treatment apparatuses of FIGS. 1A-1B, respectively, in communication with a remote device, according to embodiments of the present invention;

FIG. 3 is a diagram showing an LED-based skin treatment apparatus with a camera having an alternative optical design, according to an embodiment of the present invention;

FIGS. 4A and 4B are images of a skin lesion under differing lighting conditions;

FIG. 4C is a diagram showing a radiation-producing surface of an LED-based skin treatment apparatus having illumination LEDs, according to an embodiment of the present invention;

FIG. 4D is an image of a skin lesion taken using illumination LEDs, according to an embodiment of the present invention;

FIG. 5 is a diagram showing an LED-based skin treatment apparatus with a camera and a bezel, according to an embodiment of the present invention;

FIGS. 6A-6D are diagrams showing several operations using an LED-based skin treatment apparatus with a camera and bezel, according to an embodiment of the present invention:

FIGS. 7A-71) are diagrams showing an LED-based skin treatment apparatus with a laser projector along with exemplary laser projections, according to an embodiment of the present invention;

FIGS. 8A-8C are diagrams showing a skin lesion at different distances from an image sensor, according to an embodiment of the present invention;

FIG. 8D is a diagram showing the radiation-producing surface of FIG. 4C with selected treatment LEDs turned off, according to an embodiment of the present invention;

FIGS. 8E-8F are diagrams showing a skin lesion without and with targeted therapy, according to an embodiment of the present invention; and

FIG. 9A is a diagram showing an LED-based skin treatment apparatus with a rotating LED radiation generator, according to an embodiment of the present invention; and

FIGS. 9B-9C are diagrams showing a rotating LED radiation generator and a sample generated pattern, respectively, according to an embodiment of the present invention.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to Obscure the present invention.

U.S. application Ser. No. 17/325,203, filed May 19, 2021, discloses a system and method to evaluate the effectiveness of a skin treatment by affixing an indicator having a known color and size adjacent to a skin feature, such as a skin lesion that is being treated using a therapy, and then imaging the indicator and the skin feature together to generate before and after images.

In this application, the inventor evaluates the effectiveness of a skin treatment in a different way. Here, the inventor has developed an apparatus that combines treatment capability with imaging capability and adds several innovations to enhance the imaging results. The apparatus includes an array of treatment LEDs that treat the lesion and a camera that images the skin region containing the lesion. In one embodiment, the apparatus is configured to photograph the region of the skin containing the feature (or lesion) before and/or after the lesion treatment. This photograph may be actuated by using a button on the apparatus, a bezel attached to the apparatus to trigger the camera, a proximity or distance sensor, or a remote device. The camera may have different arrangements of optics so as to save space within the apparatus. In addition, there may be illumination LEDs that provide appropriate color balance to the images taken by the camera. There may also be a (laser) projector generating a projection comprising two or more dots or other shape that provides scale and orientation to the images. During treatment, the apparatus may be able to turn off selected treatment LEDs so as to target treatment on the skin lesion rather than on healthy skin.

The apparatus may be part of a system that includes the remote device that aids in one or more of visualizing the skin region, controlling the camera, processing and analyzing the images, and controlling the treatment LEDs.

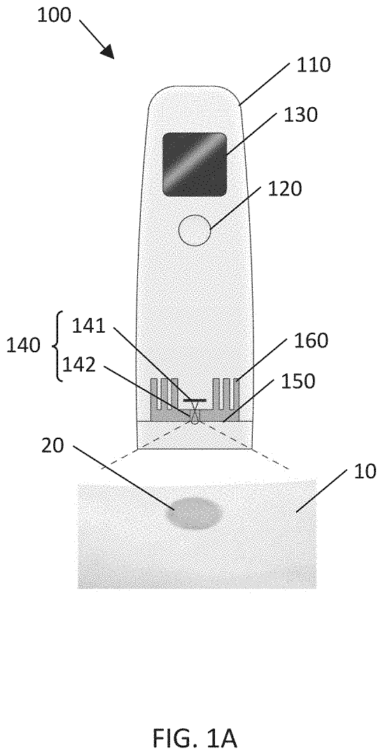

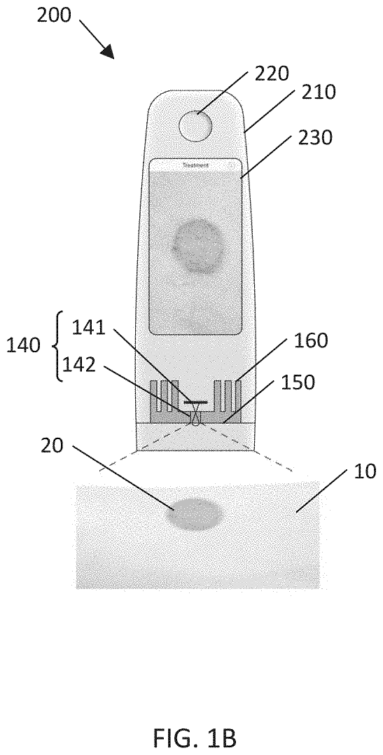

Reference is now made to FIG. 1A, which is a diagram showing an LED-based skin treatment apparatus 100 with a camera, according to an embodiment of the present invention. The apparatus includes a housing 110, a control button 120, a user interface 130, a camera 140, an LED array 150, and a heat sink 160.

Housing 110 provides the structural support for the other components of apparatus 100. Embedded in the housing are control button 120 and user interface 130. Control button 120 allows user 10 to turn on/off the apparatus. (The term “user” includes a patient using the apparatus or another person assisting that patient.) Additionally, control button 120 can be configured to allow the user to obtain an image, e.g., an image of treatment area (e.g., lesion) 20. User interface 130 may provide the user information relevant to the user's use of the apparatus. For example, the user interface can communicate to the user the time remaining in the treatment session or the battery power remaining in the device. Housing 110 may also include a controller, typically mounted on a circuit board. Such circuit board and/or controller (or processor) may include sub-circuits, for example, LED drivers to control the LED array, image processors (or sub-processors) to process and analyze the images, and circuitry to keep track of the battery functions. Software or firmware may run on the image processors or controller to process and analyze the images. The controller may be operatively coupled to camera 140, user interface 130, control button 120, and LED array 150. In one embodiment of the present invention, the controller may direct the camera to obtain an image of a region of the user's skin after the user presses control button 120.

Camera 140 includes an image sensor 141 and a lens 142. Camera 140 may also include additional components such as an image processor. Lens 142 and image sensor 141 may be vertically aligned in the center of the apparatus to provide an image of uniform size. As shown in FIG. 1A, such a configuration may require a hole in heat sink 160. Lens 142 can be shaped and sized as needed to provide an optimal image to the image sensor.

LED array 150 includes one or more treatment LEDs. The treatment LEDs may be bare die or encapsulated or any other configuration. The treatment LEDs may also be mounted on a circuit board. The treatment LEDs may transmit ultraviolet (UV) radiation (e.g., UV-A, UV-B, or narrowband UV-B) or other wavelengths, including infrared and visible, that are able to treat various skin diseases. The treatment LEDs may have a variety of configurations, which may be based on LED size and cost, housing size, manufacturability, and overall cost. For example, the LED array may be arranged in a square matrix, including 2×2, 3×3, 4×4, 5×5, etc. LEDs. Other embodiments may include different configurations of LEDs, such as rectangular, circular, or star patterns. These embodiments may include as few as one LED or another quantity of LEDs, such as 5, 10, 20, 50, etc.

Heat sink 160 is placed in thermal and/or direct contact with the LED array (or the LED array circuit board) to provide for heat dissipation of heat generated by the LED array. The heat sink can include passive technology, e.g., a plurality of fins that helps dissipate the heat from the LED array. The heat sink may include active technology, e.g., a fan or pumped liquid. The heat sink's size and configuration are based on the thermal dissipation needs of the apparatus, which are based in part on the number, configuration, size, and output of the LEDs included in the LED array.

Reference is now made to FIG. 1B, which is a diagram showing LED-based skin treatment apparatus 200 with a display screen 230, according to an embodiment of the present invention. Apparatus 200 is substantially similar to apparatus 100 except that apparatus 200 does not need to solely include user interface 130 and, instead, includes display screen 230 that makes it easier for the user to image the skin lesion. Additionally, apparatus 200 may include housing 210 that is reconfigured compared to housing 110 to accommodate the display screen. Apparatus 200 may also include control button 220 that is relocated compared to control button 120 to a position above the display screen. As described further below, apparatus 200 may also include a controller (not pictured) that performs functions in addition to those of the controller of apparatus 100.

Like user interface 130, display screen 230 may provide the user information relevant to the user's use of the apparatus. For example, display screen 230 may communicate to the user the time remaining in the treatment session or the battery power remaining in the device. Unlike user interface 130, display screen 230 may also display the image of a region of the user's skin. Display screen 230 may use LCD or OLED or any other display technology.

The controller in apparatus 200 includes the functionality described above with respect to the controller in apparatus 100. The controller in apparatus 200 may include additional functionality compared to the controller in apparatus 100 that allows apparatus 200 to transmit the image of a region of the user's skin to display screen 230,

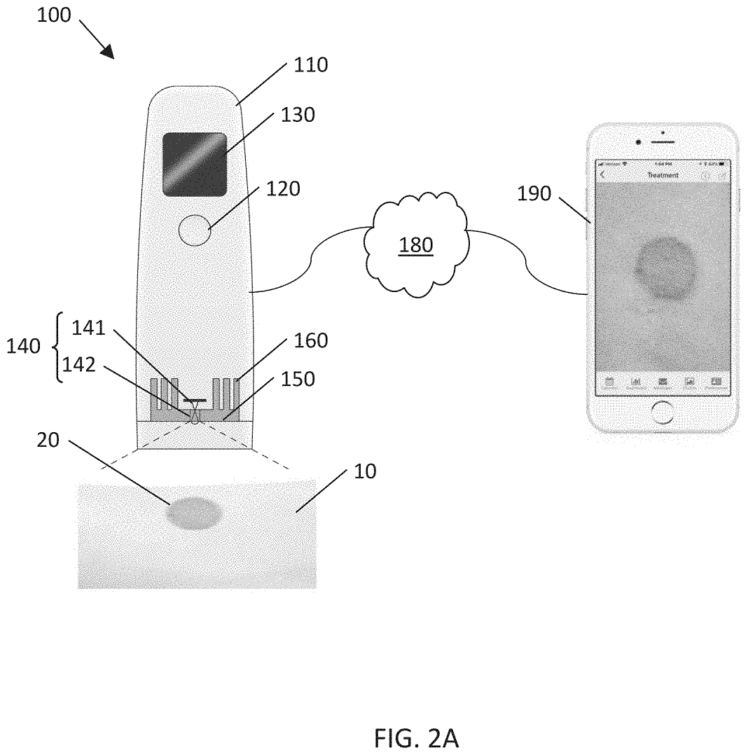

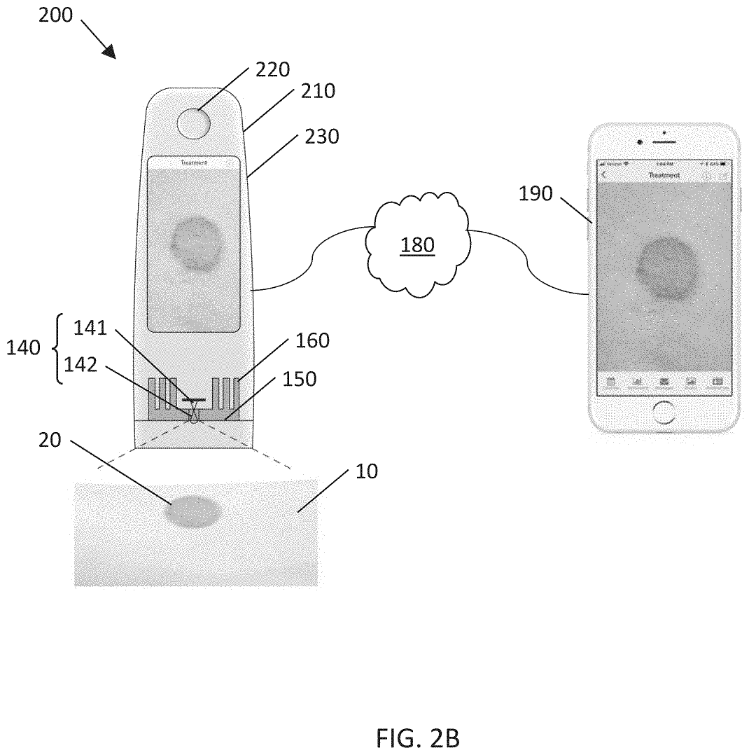

FIGS. 2A-2B are diagrams showing the LED-based skin treatment apparatuses of FIGS. 1A-1B, respectively, in communication with a remote device, according to embodiments of the present invention. Instead of using display screen 230 to display the image of the user's skin region, apparatuses 100 and 200 may be connected to remote device 190 via connection 180, so that user 10 may view the skin region on remote device 190. This may be advantageous in the situation in which the skin lesion is in a difficult-to-reach or a difficult-to-see location (e.g., back, back of the neck), and thus the user is unable to easily see display screen 230 or user interface 130 on the apparatus itself. Remote device 190 may display the functions previously, described as being displayed on user interface 130. Remote device 190 may be a smartphone or a personal digital assistant or some other portable computing device that includes a display.

Connection 180 may be a wired or a wireless connection. If wireless, it may be a Bluetooth® or Wi-Fi or other short-range connection such as a Near Field Communication (NFC) connection or a wireless local area network (LAN). In another embodiment, connection 180 may connect apparatus 100 and remote device 190 via the Internet, using Wi Fi, cellular, and/or the public-switched telephone network (PSTN).

The controller in apparatuses 100 or 200 may receive communications from remote device 190. Such communications may inform the apparatus that another image is needed and, optionally, the reason for a new image, such as the image being too dark, too light, too close to the treatment area, or too far from the treatment area. The controller then may communicate the need for another image and, optionally, the deficiencies in the image to the user via user interface 130 or display screen 230.

The controller in apparatuses 100 and 200 may also activate and deactivate the LED array to perform the LED-based skin treatment. The duration of the skin treatment can be pre-set such that every treatment provides the same dose, which may comprise the power of the dose over a certain length of time. In another embodiment, the processor allows the user to select the duration and/or dose of the treatment. The controller may display optional treatment durations and/or doses to the user on the user interface or the display screen. The user can then change the duration and/or dose and select the desired duration/dose using control buttons 120 or 220. In another embodiment, the controller may receive the treatment duration from remote device 190 based upon, for example, the severity of the skin lesion. In addition, or in the alternative, remote device 190 may run a software application, such as a “mobile app,” that controls the duration/dosing options and image processing and analysis.

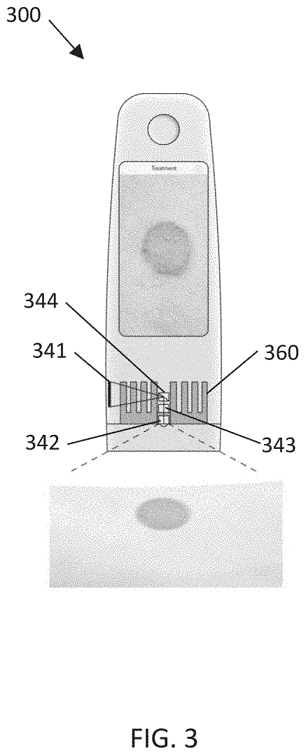

Reference is now made to FIG. 3, which is a diagram showing an LED-based skin treatment apparatus 300 with a camera having an alternative optical design, according to an embodiment of the present invention. Apparatus 300 is substantially similar to apparatus 200 except that apparatus 300 has a different camera design than apparatus 200.

As shown in FIG. 3, apparatus 300 includes heat sink 360 having a hole in the center for camera components. The camera components in the center of the heat sink include lens 342, relay or reflective optics 343, and angled relay or reflective optics 344 such as prisms and/or mirrors. Light from the region of the user's skin that is being imaged is transmitted through the lens, the relay or reflective optics, and the angled relay or reflective optics to image sensor 341, which is located external to the heat sink. While FIG. 3 shows only a single lens 342, relay optic 343, and angled relay optic 344, other embodiments can include any number of these optical relay and/or reflective components to transmit the light from the region of the user's skin that is being imaged to the image sensor.

Image sensor 341 is located external to the heat sink and is shown in a vertical orientation, so as to maximize the integrity and efficiency of the heat sink and its cooling fins (if so used). Accordingly, heat sink 360 can have a larger surface area and be more efficient compared to heat sink 160, thus allowing for improved thermal dissipation. For example, heat sink 360 may include more vertical heat dissipating fins compared to heat sink 160.

In another embodiment that is not pictured, the camera (lens and image sensor) is mounted directly on the LED array circuit board. Thus, the camera does not impinge on the heat sink at all.

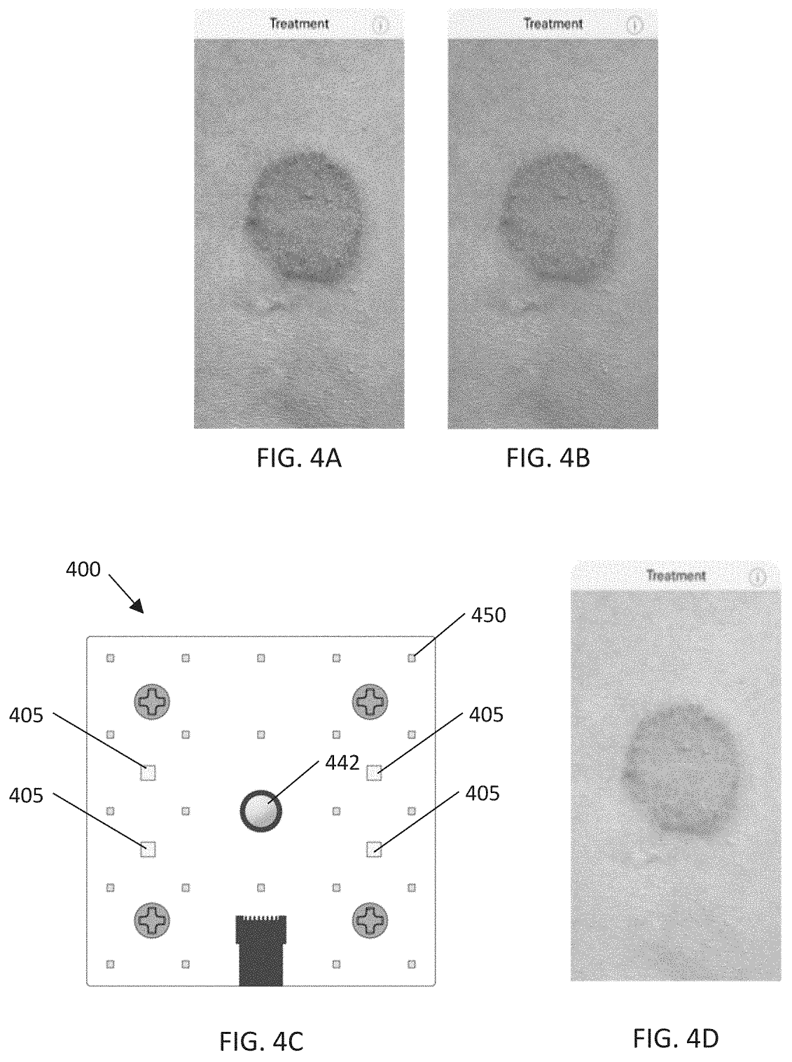

As discussed in U.S. application Ser. No. 17/325,203, images of skin lesions are often affected by poor lighting conditions. At the very least, lighting conditions may differ between before and after images of a lesion. FIGS. 4A and 4B are images of a skin lesion under differing lighting conditions. U.S. application Ser. No. 17/325,203 presents an approach to correct for such differences.

This application takes a different approach—add illumination LEDs to provide for constant and correct lighting conditions. FIG. 4C is a diagram showing a radiation-producing surface 400 of an LED-based skin treatment apparatus having illumination LEDs, according to an embodiment of the present invention. Radiation-producing surface 400 includes LED array 450, lens 442, and illumination (e.g., white light or visible light) LEDs 405, FIG. 41) is an image of a skin lesion taken using illumination LEDs, according to an embodiment of the present invention.

As shown in FIG. 4C, LED array 450 includes approximately twenty-five treatment LEDs arranged in a square-shaped grid. However, as explained with respect to LED array 150, the LED array may have a variety of configurations, which may be based on LED size and cost, housing size, manufacturability, and overall cost. For example, the LED array may be arranged in a square matrix of LEDs, including 2×2, 3×3, 4×4, 5×5, etc., or rectangular, circular, or star patterns that can include from one to many LEDs, such as 1, 5, 10, 20, 50, etc. As with LED array 150, treatment LEDs in LED array 450 may transmit UV radiation (e.g., UV-A, UV-B, or narrowband UV-B) or other wavelengths that are able to treat various skin diseases.

Lens 442 is a part of the camera and is operatively coupled to the other camera components, such as the relay or reflective optics and the image sensor. Lens 442 can be shaped and sized as needed to provide an optimal image to the image sensor.

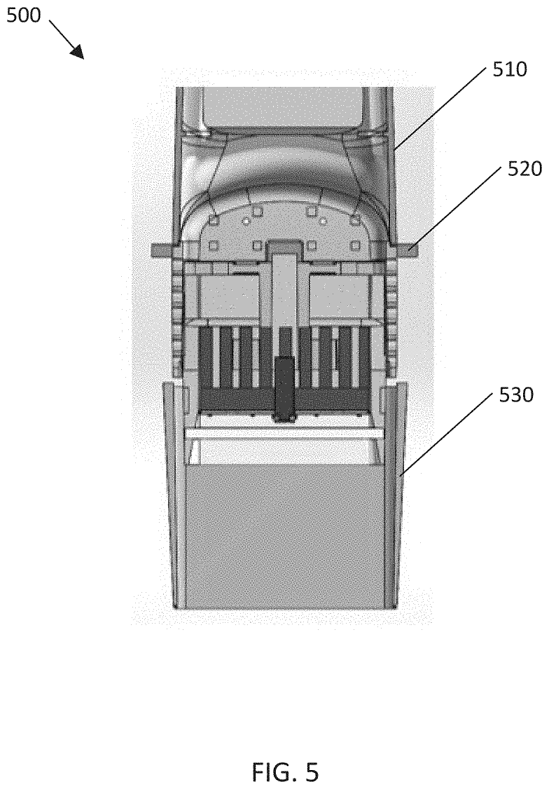

Radiation-producing surface 400 includes illumination LEDs 405 that provide improved images because they reduce inconsistent and incorrect color tint and color temperature. Four illumination LEDs are shown in FIG. 4C, but a different quantity of illumination LEDs can be used, such as any number from 1 to 10. The controller may activate the illumination LEDs prior to instructing the camera to obtain an image. The controller then may deactivate the illumination LEDs after the camera has obtained an image. Alternatively, the illumination LEDs may appear in a continuous (e.g., CW) or pulsed mode or used in a flash mode that is synchronized with the camera shutter. The light generated by the illumination LEDs should be greater than the ambient light. If the apparatus includes a bezel as described below and shown in FIG. 5, the ambient light will be minimized. However, if there is no bezel, or the bezel does not form a tight seal on the user's skin, a flash may provide the necessary amount of light. In other embodiments, the illumination LEDs are calibrated and white or color balanced prior to their arrival to the end user. Pre-calibrated illumination LEDs further minimize image problems due to inconsistent and incorrect color tint and color temperature. In other embodiments, the apparatus allows the user to perform the initial illumination LED calibration. Whether calibrated at the factory or by the user, the apparatus may allow users to perform a re-calibration of the illumination LEDs.

Reference is now made to FIG. 5, which is a diagram showing an LED-based skin treatment apparatus 500 with a camera and a bezel 530, according to an embodiment of the present invention. The bezel can be attached to any of the previously described embodiments of skin treatment apparatuses. The bezel extends approximately 26 mm beyond the bottom of the apparatus housing 510. In other embodiments, the bezel can extend a shorter or longer distance from the bottom of the apparatus housing.

The bezel is movable in a vertical direction with respect to the housing. The apparatus further includes a spring (not pictured), such that the bezel is spring-loaded to be in the extended/natural position when not in use, i.e., without any user operation of the apparatus. The apparatus with the bezel in its extended position is shown in FIG. 5. Apparatus 500 further includes bezel stops 520. The bezel stop provides a physical barrier that prevents the bezel from moving further up the housing in the vertical direction past the bezel stops.

The controller is able to track the position of the bezel with respect to the housing. The controller may use inputs from position-tracking hardware (not pictured) that are also included in the apparatus. Depending on the position of the bezel with respect to the housing, the controller may take certain actions described below to obtain the images of the region of the user's skin that is being imaged and perform the skin treatment.

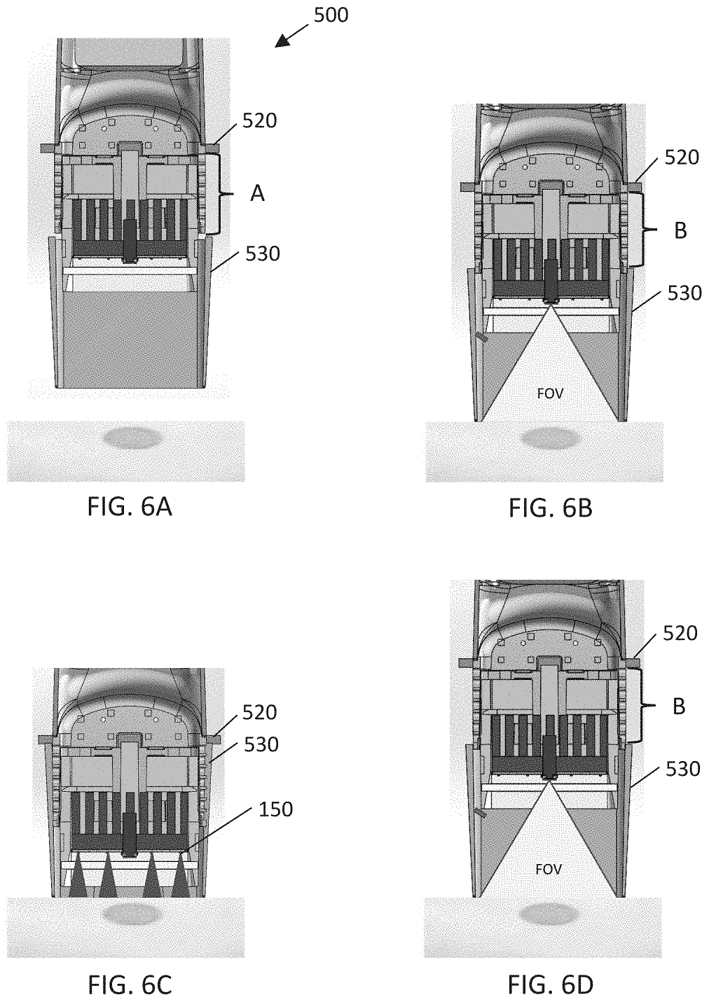

Reference is now made to FIGS. 6A-6D, which are diagrams showing several operations using an LED-based skin treatment apparatus with a camera and a bezel, according to an embodiment of the present invention. In FIG. 6A, apparatus 500 is in a first position (“Position A”) where the distance between the bezel stop and the top of the bezel is a distance A. Position A is the bezel's natural or resting state such that it is at a maximum distance from the end of the bezel stop. The bezel is maintained in Position A by the spring. In Position A, the camera is not actively obtaining images, the illumination LEDs are not activated, and the LED array is not activated.

In FIG. 6B, apparatus 500 is in a second position (“Position B”), where the distance between the bezel stop and the top of the bezel is a distance B. Position B is the position of the bezel once the user has placed the bezel against skin surrounding the treatment area and has pressed the apparatus against the skin such that pressure applied by the user forces the bezel up in the vertical direction, thus compressing the spring. In one embodiment, the bezel's distance of travel between Position A and Position B is 1-2 mm. In other embodiments, the bezel's distance of travel between Position A and Position B can be from 0.5 mm up to a few mm less than the bezel's total possible travel distance.

In Position B, the controller activates the illumination LEDs and instructs the camera to obtain an image of a region of the user's skin that is surrounded by the bezel. In other embodiments, the controller instructs the camera to obtain numerous successive images of the region of the user's skin that is surrounded by the bezel. If there were no bezel (or a very short one), the camera would be closer to the user's skin and the field of view (“FOV”) could be as much as, e.g., 135 degrees, which may result in distorted images. Such distorted images may still be usable or correctable, but are not preferred. To narrow the FOV, the optical system may be modified and then there needs to be sufficient stand-off to accommodate the narrower FOV. Using a bezel approximately 2.6 mm long accommodates an FOV of approximately 77 degrees, which is an FOV that provides an undistorted (or at least less distorted) image. The combination of the optical system and the length of the bezel to provide a specific FOV is variable, so a 26-mm-long bezel and a 77-degree FOV are exemplary. Other bezel lengths and FOVs may be used until the images become too distorted for subsequent correction and/or practical purposes. In some situations, the image taken when the bezel is very short (with a wide FOV) is also correctable. In addition, because the bezel presents a substantially flat outline to contact the skin, the camera is self-aligning and images are more repeatable.

In FIG. 6C, apparatus 500 is in a third position (“Position C”) where the top of the bezel is in contact with bezel stop 520. In Position C, the vertical movement of the bezel is stopped by the top of the bezel contacting the bezel stop. The distance moved may be, for example, 10-14 mm or approximately half the length of the bezel. In Position C, the controller deactivates (or does not activate) the illumination LEDs and activates the LED array to commence the skin treatment. The controller deactivates the LED array after the skin treatment is completed. In other embodiments, the controller may also deactivate the LED array if the bezel moves from Position C, such as may occur if the user lifts the apparatus from the skin. In other embodiments, the controller can deactivate the illumination LEDs when the bezel is somewhere between Positions B and C.

In FIG. 6D, apparatus 500 is in a fourth position (“Position D”) where the distance between the bezel stop and the top of the bezel is a distance D. In Position D, the controller activates the illumination LEDs and instructs the camera to obtain an image of a region of the user's skin that is surrounded by the bezel. In other embodiments, the controller instructs the camera to obtain numerous successive images of the region of the user's skin that is surrounded by the bezel. In one embodiment, Position B equals Position D, which provides consistency between the pre-treatment image obtained at Position B and the post-treatment image obtained at Position D. The controller deactivates the illumination LEDs once it senses that the bezel has returned to Position A, which is the bezel's natural or resting state. Below is a table showing the positions and the states of the treatment LEDs, the illumination LEDs, and the camera, along with the motion of the bezel:

| | Treatment | Illumination | | |
|---|---|---|---|---|
| Position | LEDs | LEDs | Camera | Bezel Motion |
| A | Off | Off | Off | Still/rest |
| B | Off | On | On | Downward |
| C | On | Off | Off | Stopped by stop |
| D | Off | On | On | Upward |

In another embodiment, instead of a mechanical stop there may be an optical proximity or distance sensor on radiation-producing surface 400 that detects the distance between the LED array and the skin and can activate the camera at the right distance. Such an arrangement may address the problem of skin possibly bulging into the bezel, so it may more accurately measure the distance to provide more consistency for taking and interpreting before and after images.

Such proximity sensor may also be used to gauge the distance for lesion treatment. This arrangement is able to prevent the treatment LEDs from getting too close to the skin, and so can reduce the power of the treatment LEDs or turn them off completely if too close. This arrangement can also increase the power of the treatment LEDs if they are too further from the skin. This arrangement may also signal to the user that the apparatus is too close or too far from the skin (or just the right distance).

In another embodiment, once the bezel is in treatment position and if the treatment position is the same as the picture-taking position, the timing of the illumination LEDs and treatment LEDs is performed in software. No additional switches are needed in this mode.

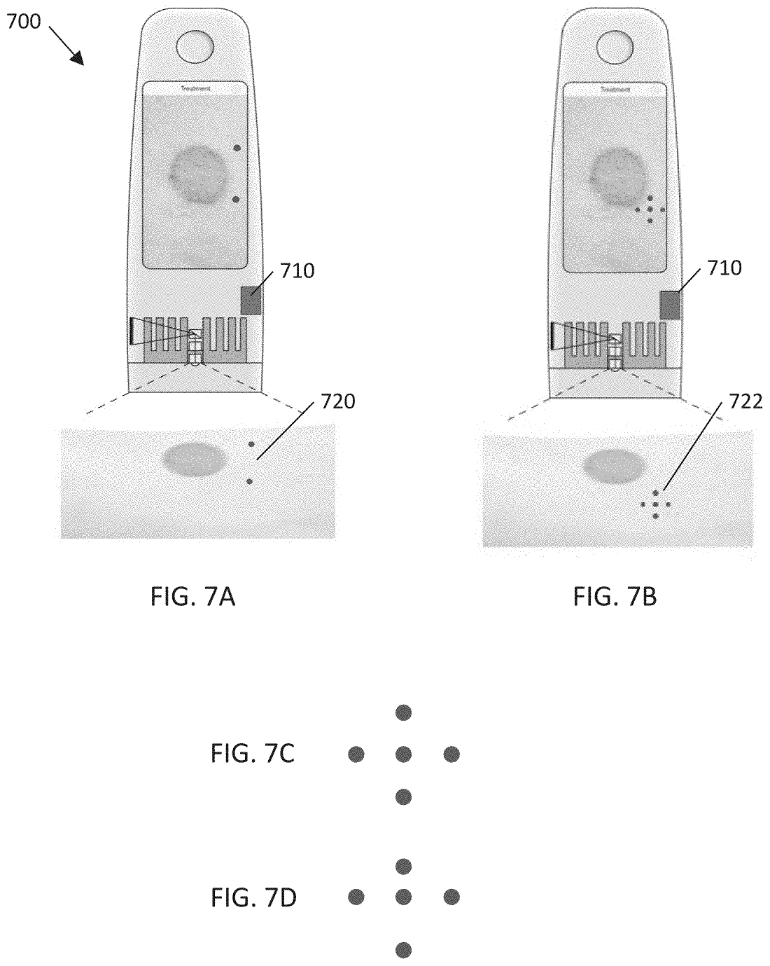

Reference is now made to FIGS. 7A-7D, which are diagrams showing an LED-based skin treatment apparatus 700 with a laser projector 710 along with exemplary laser projections, according to an embodiment of the present invention. A laser projection provides consistent scale and orientation to the images taken by the camera. A laser projector is preferred because it provides one or more collimated beams that diverge very little over the distances contemplated by the apparatus. In the embodiment shown in FIG. 7A, laser projector 710 projects two parallel laser dots 720 on the user's skin adjacent to or on the lesion. Because of the collimation, these dots are always substantially the same distance apart. Alternatively, the laser projector could project more than two dots or a collimated bar or circle or other projection having a consistent shape and consistent size. The laser projection provides the camera with a reference measurement. In the embodiment shown in FIG. 7B, laser projector 710 projects a five-dot pattern 722 on the user's skin adjacent to or on the lesion. In addition to the reference measurement provided by any of the laser projections generated by laser projector 710, the five-dot pattern also provides information as to whether or not the apparatus is normal to the skin's surface, such that the apparatus is at the proper angle for imaging the treatment area, i.e., that the camera is directly above and looking straight down at the treatment area without any skew or distortion. For example, as shown in FIG. 7C, if the center dot of the five-dot pattern is equidistant from the other four dots, then the user knows that the camera is at the correct position for imaging the treatment area. However, if the center dot is closer to any of the other four dots, as shown in FIG. 7D, then the user knows that the image is skewed and that it would be preferable to straighten the apparatus/camera. Even if there is some skew, the amount of skew can be used to correct the image when it is processed by the apparatus's or mobile app's software or firmware. In another embodiment, the apparatus uses the laser dot pattern to activate the camera only when the dots are in proper alignment.

Although laser projector 710 is called a “laser” projector, it may include non-laser devices if the output of the projector is substantially collimated over the distances contemplated by the invention. In addition, while the laser projectors in FIGS. 7A-7B are shown located within the embodiment of the apparatus shown in FIG. 3, the laser projector can be located within any embodiment of the apparatus of the present invention, including those shown in FIGS. 1A-2B, 3, 5, 6A-6D, and 9.

Another innovation in this application is the use of image stitching. The treatment and image area of apparatuses 100-300 and the other apparatuses described herein is often smaller than the area of the skin lesion, or at least one of the dimensions of the skin lesion is greater than the dimensions of the treatment and image area of the apparatuses. For example, the treatment and image area may be 2 inches×2 inches, but a skin lesion may cover parts of a rectangle 5 inches long×3 inches wide. Stitching allows images of parts of a skin lesion to be taken and then combined into a single image showing the whole lesion. Using the bezel-operated camera and the illumination LEDs, a user is able to take multiple, sequential images of a skin lesion. Stitching software in the controller or the remote device can stitch the multiple images into a single image.

The camera has been described up to this point as including a lens and an image sensor. Some embodiments of this camera may be a still camera and other embodiments may be a video camera. The stitching software may combine still images of the skin lesion as well as screenshots from video images of the skin lesion. The camera may include position sensing to sense the position of the apparatus as it moves across the lesion. Thus, the camera may automatically take one or more images as the handpiece moves.

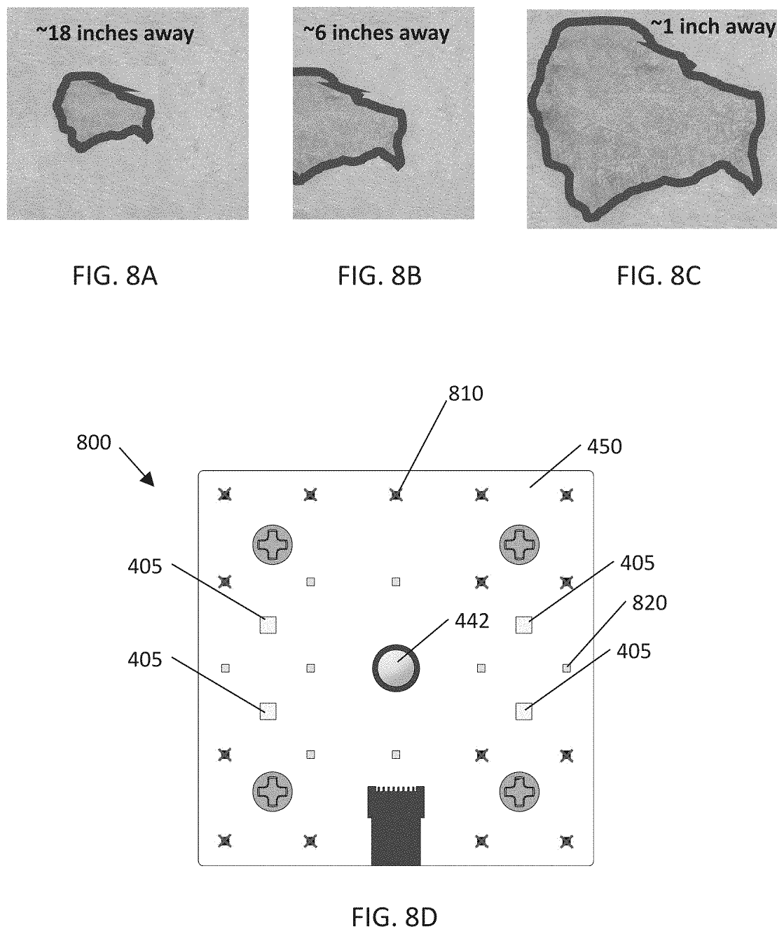

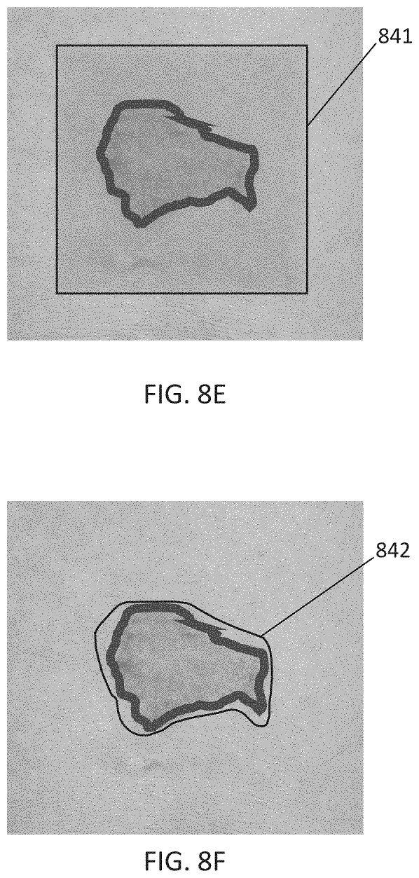

The inventor has also recognized that the arrangement of the treatment LEDs shown in the embodiments in FIG. 4C may not be optimal, because the radiation pattern is square, but skin lesions often are irregularly shaped, thus exposing healthy skin to unneeded radiation treatment. In response, during treatment, the apparatus may turn off selected treatment LEDs so as to target treatment on the skin lesion rather than on healthy skin. Reference is now made to FIGS. SA-8C, which are diagrams showing a skin lesion at different distances from an image sensor, according to an embodiment of the present invention. FIG. 8A shows what the image sensor may see from about 18 inches away (although in reality the lesion may be out of focus, which is not pictured). As the apparatus moves closer, the lesioned area may come into better focus and into the field of view. FIG. 8B shows what the image sensor may see from about 6 inches away, where some of the lesion may be outside of the field of view and slightly out of focus (again, lack of focus is not pictured). Finally, FIG. 8C shows what the image sensor may see from about 1 inch away (which may be the treatment distance)—a well-focused image within the field of view.

Reference is now made to FIG. 8D, which is a diagram showing the radiation producing surface of FIG. 4C (indicated by 800) with selected treatment LEDs turned off to approximate the shape of the skin lesion, according to an embodiment of the present invention. More generally, the selective treatment process of the invention allows a subset of treatment LEDs to be used to focus the treatment on the skin lesion itself, rather than applying LED radiation to healthy skin. In one embodiment, the controller determines which subset of treatment LEDs of the LED array to turn on (activate) during the skin treatment based on the geometry and quantity of the treatment LEDs in the LED array. The controller's determination of which treatment LEDs to turn on may also be based on the particular geometry and size of the lesion or treatment area. The controller may perform pattern recognition to determine the areas that are eligible for skin treatment. For example, as shown in FIG. 8D, only LEDs 820 that are over the treatment area are energized or activated, while LEDs 810 that are not over the treatment area will not be energized during the skin treatment. FIG. 8E shows the skin lesion without targeted therapy, and FIG. 8F shows the skin lesion with targeted therapy. In FIG. 8E, there is a good amount of healthy skin within outline 841 that has been treated with the LEDs and thus has been discolored and/or damaged. In FIG. 8F, outline 842 approximates the treated area. While the selective treatment process is described with respect to the exemplary radiation-producing surface 400 and its particular configuration of LED array 450, lens 442, and illumination LEDs 405, as discussed above, the radiation-producing surface can include a variety of configurations for the LED array, lens, and illumination LEDs.

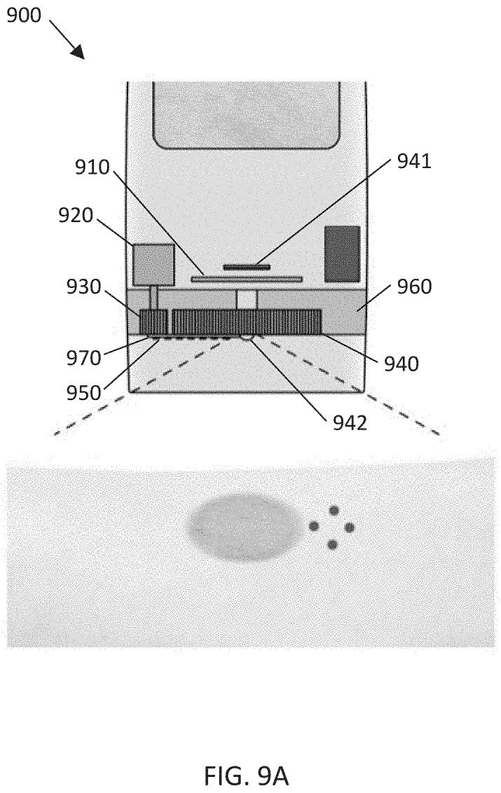

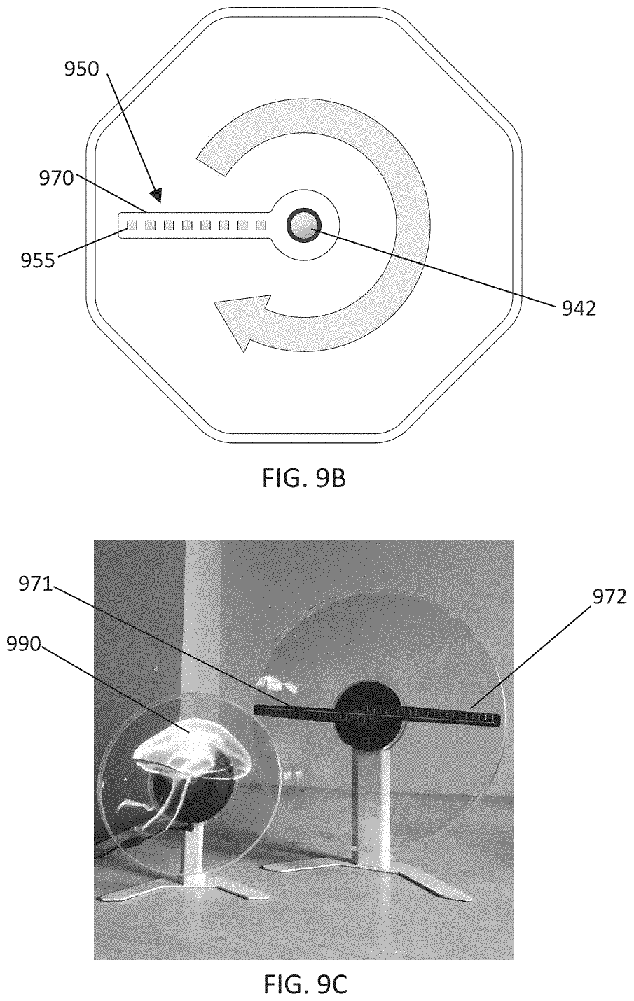

The arrangement of the LEDs in FIGS. 4 and 8 are not the only ones that may be used. Reference is now made to FIG. 9A, which is a diagram showing an LED-based skin treatment apparatus 900 with a rotating LED radiation generator, and FIGS. 9B-9C, which are diagrams showing a rotating LED radiation generator and a sample generated pattern, respectively, according to an embodiment of the present invention. Like the previous embodiments discussed above, the embodiment of FIG. 9A includes the housing, the control button (not pictured), the display screen, image sensor 941, lens 942, and the controller (not pictured).

Instead of array 150 or 450 as shown previously the embodiment of FIG. 9A includes a linear LED array 950 that spins on a wheel surrounding lens 942, as shown in FIG. 9B. LED array 950 includes treatment LEDs 955 disposed on LED assembly arm 970. LED assembly arm 970 may be shaped as an elongated blade of uniform width that extends from the center of the apparatus, as shown in FIG. 9B. However, the LED assembly arm can be of any shape, such as an elongated blade where the width is greater on the end farthest away from the center of the apparatus. The LED array may be a single row of treatment LEDs spanning the entire length of the LED assembly arm, as shown in FIG. 9B. In other embodiments, the array may include more than one row. There may also be two parallel linear arrays, 971, 972, as shown in FIG. 9C. FIG. 9C also shows a sample pattern 990 that the spinning linear arrays can generate. In another embodiment, instead of a spinning bar, there may be a bar that moves vertically or horizontally to cover the treatment area.

FIG. 9A shows how LEI) array 950 fits within apparatus 900. LED array 950 is attached to transfer gear 940, which engages drive gear 930, which is driven by motor 920. Also included in apparatus 900 is a rotary encoder 910 and a water-cooled heat sink 960. The controller controls the rate that LED assembly arm 970 rotates via instructions sent to motor 920. Based on these instructions, the motor rotates its shaft at the instructed rate. The motor's shaft is connected to drive gear 930, which then rotates as the same rate as the motor's shaft. The drive gear is operatively coupled to transfer gear 940 via gear teeth on each respective gear. Based on the size of the gears, the transfer gear rotates at a rate that is proportional to the motor shaft's rate of rotation. The transfer gear rotates about a center axis that typically lies along the center line of the apparatus. Rotary encoder 910 is used to keep track of the position of the arm.

The controller implements a selective treatment process, where it determines which LEDs of the LED array to activate and for how long based on the rate of rotation of the motor during the skin treatment so as to minimize the application of LED-based radiation to areas of healthy skin. The controller determines which individual treatment LEDs should be turned on based on the geometry and quantity of the treatment LEDs in the LEI) array in addition to the geometry and configuration of the gears connecting the motor to the LED assembly arm. The controller's determination of which treatment LEDs to turn on and the motor's rotation rate may also be based on the particular geometry and size of the treatment area.

Water-cooled heat sink 960 is placed in thermal contact with the LED array to dissipate heat generated by the LED array. The heat sink's size and configuration are based on the thermal dissipation needs of the apparatus, which are based in part on the quantity, configuration, size, efficiency, and output of the treatment LEDs included in the LED array.

In sum, an apparatus is presented that combines skin treatment capability with imaging capability and adds several innovations to enhance the imaging results. There is a display screen with which to view the image before photographing, and the apparatus can be connected to a remote device on which to view the image. The photograph can be taken automatically before or after a treatment session. The apparatus includes illumination LEDs to provide appropriate color balance to the images taken to assist in comparison of before and after images. The apparatus also includes laser projections to provide scale and orientation to the images to assist in comparison of before and after images. The camera has different arrangements of optics to save space within the apparatus so that heat sink space can be maximized. Various heat sink arrangements are also presented. In addition, during treatment, the apparatus is able to target treatment on the skin lesion rather than on skin by activating selected treatment LEDs.

An apparatus for treating a lesion within a skin region may include some or all of the following aspects:
An LED array configured to treat the lesion, where the LED array includes one or more LEDs.
A camera capable of imaging the skin region, where the camera includes an image sensor and a lens.
The apparatus is configured to automatically photograph the skin region before or after or before and after the lesion treatment.
The apparatus may include a bezel. The bezel may actuate the camera to perform the automatic photography. The bezel may be long to accommodate a narrow field of view. The bezel may self-align the camera. An optical proximity or distance sensor may be used to facilitate the automatic photography.
The camera may be self-aligned. The apparatus may include a display screen to show the image. The LEDs may be UV, UV-A, UV-B, narrowband UV-B, infrared, or visible.
The apparatus may include illumination LEDs. The camera may include angled relay or reflective optics. The apparatus may include a laser projector or a non-laser projector. The apparatus may connect to a remote device to perform the automatic or non-automatic photography.
The camera may be still or video. The apparatus may include software capable of stitching still images or video images together.
The apparatus may also include a heat sink, which may be made of fins or may be water cooled.
The apparatus may be used to target treatment to the shape of the lesion. This may be done by activating certain LEDs in a square or rectangular or circular array. This may also be done using a linear array on a spinning arm and activating the LEDs at the correct time to treat the lesion area.

Aspects of the present invention may be embodied in the form of a system, a computer program product, or a method. Similarly, aspects of the present invention may be embodied as hardware, software, or a combination of both. Aspects of the present invention may be embodied as a computer program product saved on one or more computer-readable media in the form of computer-readable program code embodied thereon.

The computer-readable medium may be a computer-readable storage medium. A computer-readable storage medium may be, for example, an electronic, optical, magnetic, electromagnetic, infrared, or semiconductor system, apparatus, or device, or any combination thereof.

Computer program code in embodiments of the present invention may be written in any suitable programming language. The program code may execute on a single computer, or on a plurality of computers. The computer may include a processing unit in communication with a computer-usable medium, where the computer-usable medium contains a set of instructions, and where the processing unit is designed to carry out the set of instructions.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. An apparatus for treating a lesion within a skin region, comprising:
a light emitting diode (LED) array configured to treat the lesion, the LED array comprising one or more LEDs; and
a camera capable of imaging the skin region, the camera comprising an image sensor and a lens;
wherein the apparatus is configured to automatically photograph the skin region before or after or before and after the lesion treatment.

2. The apparatus of claim 1, further comprising a bezel configured to actuate the camera to perform the automatic photography.

3. The apparatus of claim 2, wherein the field of view of the camera is narrowed.

4. The apparatus of claim 2, wherein the bezel self-aligns the camera.

5. The apparatus of claim 1, further comprising a mechanism to self-align the camera.

6. The apparatus of claim 1, further comprising a display screen to display an image of the lesion in real time.

7. The apparatus of claim 1, wherein the LEDs transmit ultraviolet radiation.

8. The apparatus of claim 7, wherein the LEDs transmit narrowband ultraviolet-B radiation.

9. The apparatus of claim 1, further comprising an illumination LED within the LED array.

10. The apparatus of claim 1, wherein the image sensor is positioned parallel to the plane of the LED array and directly above or behind the lens.

11. The apparatus of claim 1, further comprising relay or reflective optics between the lens and the image sensor, wherein the image sensor is positioned at an angle to the plane of the LED array.

12. The apparatus of claim 1, further comprising a projector configured to project a pattern onto or adjacent the skin region.

13. The apparatus of claim 12, wherein the pattern comprises two laser dots projected in a parallel fashion from the projector.

14. The apparatus of claim 12, wherein the pattern comprises five laser dots and indicates whether the apparatus is normal to the skin region.

15. The apparatus of claim 12, wherein the pattern is used to indicate the size of the lesion.

16. The apparatus of claim 12, wherein the projector is a non-laser projector.

17. The apparatus of claim 1, further comprising a remote device configured to perform the automatic photography.

18. The apparatus of claim 1, wherein the camera photographs multiple images and stitches them together into a single image.

19. The apparatus of claim 1, wherein the camera comprises a video camera.

20. The apparatus of claim 19, wherein multiple images taken from the video camera are stitched together into a single image.

21. The apparatus of claim 1, wherein the LEDs in the LED array are selectively activated to substantially target treatment to the shape of the lesion.

22. The apparatus of claim 1, wherein the LED array is disposed on a wheel and the LEDs in the LED array are selectively activated to substantially target treatment to the shape of the lesion.

23. An apparatus for treating a lesion within a skin region, comprising:
a light emitting diode (LED) array configured to treat the lesion, the LED array comprising one or more LEDs;
a camera capable of imaging the skin region, the camera comprising an image sensor and a lens; and
a bezel configured to actuate the camera to automatically photograph the skin region before or after or before and after the lesion treatment.

24. The apparatus of claim 23, wherein the LEDs in the LED array are selectively activated to substantially target treatment to the shape of the lesion.

25. A system for treating a lesion within a skin region, comprising:
a treatment apparatus comprising:
a light emitting diode (LED) array configured to treat the lesion, the LED array comprising one or more LEDs; and
a camera capable of imaging the skin region, the camera comprising an image sensor and a lens; and
a remote device configured to photograph the skin region before or after or before and after the lesion treatment.

26. The system of claim 25, wherein the remote device processes and analyzes a photographed image.

27. The system of claim 25, wherein the treatment apparatus further comprises a controller, and the controller processes and analyzes a photographed image.

28. The apparatus of claim 3, wherein the length of the bezel is designed to accommodate the narrowed field of view of the camera.

29. The apparatus of claim 10, wherein the illumination LED transmits visible light.

30. The apparatus of claim 12, wherein the pattern comprises a plurality of laser dots and indicates whether the apparatus is normal to the skin region.

31. The apparatus of claim 23, further comprising relay or reflective optics between the lens and the image sensor.

32. The apparatus of claim 23, wherein the length of the bezel is designed to accommodate a narrowed field of view of the camera.

33. The system of claim 25, further comprising relay or reflective optics between the lens and the image sensor.
